(19) United States
(12) Patent Application Publication
(10) Pub. No.: US 2020/0048595 A1
(43) Pub. Date: Feb. 13, 2020

(54) EFFICIENT PRODUCTION OF BIOETHANOL IN MOBILE REACTORS

(71) Applicant: LOCUS IP COMPANY, LLC, SOLON, OH (US)

(72) Inventors: SEAN FARMER, NORTH MIAMI BEACH, FL (US); KEN ALIBEK, SOLON, OH (US)

(21) Appl. No.: 16/495,922

(22) PCT Filed: Apr. 9, 2018
(86) PCT No.: PCT/US2018/026729

§ 371 Date: Sep. 20, 2019

Related U.S. Application Data
(60) Provisional application No. 62/483,427, filed on Apr. 9, 2017.

(51) Int. Cl. C12M 1/00; C12M 1/12; C12P 7/06

(52) U.S. Cl. CPC C12M 21/12; C12M 23/06; C12P 7/06; C12M 25/00

(57) ABSTRACT

The subject invention provides systems and methods for producing bioethanol. More specifically, the present invention includes biological reactors, equipment, and materials for converting carbohydrate sources into alcohol products for use as biofuels and/or sources of electricity in, for example, remote areas.

### CROSS-REFERENCE TO RELATED APPLICATION

This applications claims the benefit of U.S. provisional patent application Ser. No. 62/483,427, filed Apr. 9, 2017, which is incorporated herein by reference in its entirety.

### BACKGROUND OF THE INVENTION

Cultivation of microorganisms, such as bacteria, yeast and fungi, is important for the production of a wide variety of useful bio-preparations. Microorganisms play crucial roles in, for example, food industries, pharmaceuticals, agriculture, mining, environmental remediation, and waste management.

There exists an enormous potential for the use of microorganisms in a broad range of industries, in particular, the use of yeast strains in the production of ethanol. Ethanol is a valuable form of energy, and can be used for generating electricity and as an alternative or supplement to, for example, petroleum-based fuels. Ethanol is also the primary source of alcohol in alcoholic beverages.

The environmental advantages of using ethanol as a fuel or energy source are wide ranging; notably, ethanol fuel produces fewer quantities of pollutants, greenhouse gas emissions and other harmful byproducts of burning fossil fuels. Furthermore, ethanol can be produced using renewable sources, such as agricultural feedstocks. When ethanol is produced from crops, such as, for example, sugarcane, potatoes, or corn, it is often referred to as “bioethanol.”

In addition to its environmental benefits, ethanol fuel also has the potential to be useful as an energy source in geographic areas where transporting fossil fuels is costly or otherwise difficult. For example, it can be difficult to provide energy to people living in rural areas due to, for example, the challenges of running transmission lines from power plants located in distant, more industrialized areas. Additionally, it can be difficult to provide energy to armed forces who are deployed and/or located in remote areas. Not only can transporting gasoline to such locations cost from $150 to $1400 per barrel, but using, for example, tanker trucks to transport the fuel creates conspicuous moving targets and increases the risks of exposing troops and their bases to enemy forces.

Using bioethanol as a fuel or energy source for situations where transporting fuel is costly and/or dangerous could help meet energy needs in those places without compromising safety, ease of access and costs. Most advantageous would be systems and methods for efficient ethanol production that can be operated locally, remotely, and securely using indigenous, readily accessible resources.

### SUMMARY OF THE INVENTION

The present invention relates to methods and portable systems for producing ethanol that can be used, for example, as a fuel or electricity source in remote areas. Specifically, the subject invention provides methods and materials for efficient use of microorganisms for producing bioethanol, as well as portable systems for such uses.

In preferred embodiments, the subject invention provides a portable system for the production of bioethanol, wherein the entire system can be housed and transported in a 10 to 50 square foot, or 20 to 40 square foot, protective container. Advantageously, the systems of the present invention can be scaled depending on the intended use.

Additionally, the portable systems can be operated at or near a site where ethanol is needed, for example, not more than 50 miles away from a site where electricity or fuel is needed. Furthermore, the protective container that houses the system can be, for example, fire- or explosion-proof, for operation at military bases, or other locations where fuel demand is high but cost, safety and security are significant concerns. Even further, the systems can be useful in rural or secluded areas, where transmission of power is difficult due to the remoteness of the area or the distance from the nearest power plant. For example, the systems can be used to produce ethanol for use in local or household generation of electricity in rural cities and towns.

In one embodiment, the portable systems of the present invention provide biological reactors for continuous conversion of carbohydrates into ethanol. In some embodiments where scaling up is desired, the system can comprise a series of biological reactors that can operate simultaneously.

In certain embodiments, the systems utilize ethanologenic organisms to convert carbohydrates into ethanol. In preferred embodiments, the systems utilize Wickerhamomyces anomalus or Saccharomyces cerevisiae yeasts, immobilized in and/or on a bead or some other medium for immobilizing yeasts in a resting state.

In certain embodiments, the biological reactors of the subject systems can comprise a column, wherein the column is attached to a feed tank containing a mixture of a carbohydrate and water. The column can be any known column having a high vertical to horizontal ratio, for example, a tube of a Winogradsky column.

In preferred embodiments, the column is loaded with immobilized yeast cells. Preferably, the yeast cells are immobilized in and/or on alginate beads. Pressure is then used to continuously transfer the carbohydrate-water mixture from the feed tank, through a tube or pipe, into the column, and over the immobilized yeast, so as to, preferably, achieve a consistent dilution rate throughout the conversion process. In specific embodiments, the carbohydrate is a locally-derived source of glucose or sucrose.

In one embodiment, the carbohydrate-water mixture flows into the column through a tube or pipe at the bottom of the column and flows out through a tube or pipe at the top. In one embodiment, the carbohydrate-water mixture flows into the column through a tube or pipe at the top of the column and flows out through a tube or pipe at the bottom.

In one embodiment, the system utilizes gravity to transfer the carbohydrate-water mixture from the feed tank and through the column, wherein the feed tank can be in an elevated position relative to the column.

In another embodiment, the system utilizes a pump to transfer the carbohydrate-water mixture. The pump can be, for example, a dosing pump, a peristaltic pump, or a centrifugal pump. The size of the pump can be scaled so as to achieve a desired and consistent dilution rate, depending on the size of the column and/or the amount of liquid therein.

In certain embodiments, the system can further comprise an apparatus for distilling and collecting ethanol from the end products of the conversion process. The apparatus can be a distiller, a still, a beer column, or any other system known in the art for purifying alcohol. Preferably, the end product is delivered directly from the column to the distilling apparatus using, for example, piping or tubing. Alternatively, the end product can be placed in a collection tank prior to being distilled.

In some embodiments, the system of the subject invention can be powered primarily by diesel generators located, for example, in trucks used to carry the contained system from point to point. In some embodiments, solar panels can be installed on top of, or otherwise near, the housing container to provide supplemental energy. In yet another embodiment, supplemental energy can be provided by wind turbines, which can also be portable.

In one embodiment, methods are provided for producing bioethanol using the systems according to the subject invention. Ethanol produced according to the subject methods can be used to supplement existing fuel sources, for example, as an additive to gasoline. Additionally, the ethanol can be burned in combustion generators to produce electricity.

In a specific embodiment, the method comprises loading an ethanologenic microorganism that has been immobilized, into a column of the subject system; mixing water and a carbohydrate in a feed tank that is attached to the column; and using a pumping apparatus, or gravity, to continuously transfer the water and carbohydrate mixture from the feed tank and over the immobilized microorganism at a consistent dilution rate, wherein the system is operated for an appropriate amount of time to produce an end product comprising 6-15 g/L of ethanol.

The method can further comprise distilling the end product to a distilled alcohol product having at least 50%, 60%, 70%, 80%, or 90% (or any percentage therebetween) alcohol by volume.

In one embodiment, the microorganism is immobilized in and/or on alginate beads. In one embodiment, the microorganism is immobilized in the pores and on the surfaces of microporous, sterile beads made of glass or plastic. In one embodiment, the microorganisms are immobilized onto a line or fiber suspended from the top of the column to the bottom of the column. In certain embodiments, the substrate to which the yeast is immobilized has been functionalized with an antibody, or other linker, to help facilitate immobilization, yet maintained biological activity, of the yeast.

Preferably, the continuous circulation of liquid into and out of the column allows for the continual removal of the ethanol-containing end product so as not to exceed a concentration of 6-15 g/L. Advantageously, this reduces growth inhibition of the microorganisms by the ethanol and facilitates continuous operation of the system.

Advantageously, the subject invention allows for continuous, uninterrupted production of yeast by-products over extended periods of time. For example, the biological reactors can be operated continuously, 24 hours a day, for several days or even months at a time. This is, in part, due to high yeast survival rates. For example, a yeast survival rate of 95% over the course of one month can be achieved using the subject systems, thus reducing the number of times the system must be re-loaded with yeast cells.

In some embodiments, wherein the system comprises a plurality of columns, the system can facilitate continuous operation by replacing a single column at a time when the microorganisms have reached the end of their ethanologenic potential. The columns can be pre-loaded with immobilized microbes in order to facilitate quick and easy replacement of the columns.

Advantageously, the methods and systems of the subject invention reduce the capital and labor costs of producing ethanol on a large scale. The subject invention provides a conversion method that not only substantially increases the yield of ethanol but simplifies production and facilitates portability. Portability can result in significant cost savings as ethanolic compositions can be produced at, or near, the site of intended use. This means that the final ethanol product can be manufactured on-site using locally-sourced materials, thereby reducing shipping costs.

### DETAILED DESCRIPTION OF THE INVENTION

The present invention relates to methods and portable systems for producing ethanol that can be used, for example, as a fuel or electricity source in remote areas. Specifically, the subject invention provides methods and materials for efficient use of microorganisms for producing bioethanol, as well as portable systems for such uses.

The concentration of ethanol produced according to the subject invention can be, for example, greater than 50%, 60%, 70%, 80%, 90%, 95%, or 99%.

In preferred embodiments, the subject invention provides a portable system for the production of bioethanol, wherein the entire system can be housed and transported in a 10 to 50 square foot, or 20 to 40 square foot, protective container. Advantageously, the systems of the present invention can be scaled depending on the intended use.

Additionally, the portable systems can be operated at or near a site where ethanol is needed, for example, not more than 50 miles away from a site where electricity or fuel is needed. Furthermore, the protective container that houses the system can be, for example, fire- or explosion-proof, for operation at military bases, or other locations where fuel demand is high but cost, safety and security are significant concerns. Even further, the systems can be useful in rural or secluded areas, where transmission of power is difficult due to the remoteness of the area or the distance from the nearest power plant. For example, the systems can be used to produce ethanol for use in local or household generation of electricity in rural cities and towns.

In one embodiment, the portable systems of the present invention provide biological reactors for continuous conversion of carbohydrates into ethanol. In some embodiments where scaling up is desired, the system can comprise a series of biological reactors that can operate simultaneously.

In certain embodiments, the systems utilizes ethanologenic organisms to convert carbohydrates into ethanol. In preferred embodiments, the systems utilize Wickerhamomyces anomalus or Saccharomyces cerevisiae yeasts, immobilized in and/or on a bead or some other substrate for immobilizing yeasts in a resting state.

In certain embodiments, the biological reactors of the subject systems can comprise a column, wherein the column is attached to a feed tank containing a mixture of a carbohydrate and water. The column can be any known column having a high vertical to horizontal ratio, for example, a tube of a Winogradsky column.

In preferred embodiments, the column is loaded with immobilized yeast cells. Preferably, the yeast cells are immobilized in and/or on alginate beads. Pressure is then used to continuously transfer the carbohydrate-water mixture from the feed tank, through a tube or pipe, into the column, and over the immobilized yeast, so as to, preferably, achieve a consistent dilution rate throughout the conversion process. In specific embodiments, the carbohydrate is a locally-derived source of glucose or sucrose.

In one embodiment, the carbohydrate-water mixture flows into the column through a tube or pipe at the bottom of the column and flows out through a tube or pipe at the top. In one embodiment, the carbohydrate-water mixture flows into the column through a tube or pipe at the top of the column and flows out through a tube or pipe at the bottom.

In one embodiment, the system utilizes gravity to transfer the carbohydrate-water mixture from the feed tank and through the column, wherein the feed tank can be in an elevated position relative to the column.

In another embodiment, the system utilizes a pump to transfer the carbohydrate-water mixture. The pump can be, for example, a dosing pump, a peristaltic pump, or a centrifugal pump. The size of the pump can be scaled so as to achieve a desired and consistent dilution rate, depending on the size of the column and/or the amount of liquid therein.

In certain embodiments, the system can further comprise an apparatus for distilling and collecting ethanol from the end products of the conversion process. The apparatus can be a distiller, a still, a beer column, or any other system known in the art for purifying alcohol. Preferably, the end product is delivered directly from the column to the distilling apparatus using, for example, piping or tubing. Alternatively, the end product can be placed in a collection tank prior to being distilled.

In some embodiments, the system of the subject invention can be powered primarily by diesel generators located, for example, in trucks used to carry the contained system from point to point. In some embodiments, solar panels can be installed on top of, or otherwise near, the housing container to provide supplemental energy. In yet another embodiment, supplemental energy can be provided by wind turbines, which can also be portable.

In one embodiment, methods are provided for producing bioethanol using the systems according to the subject invention. Ethanol produced according to the subject methods can be used to supplement existing fuel sources, for example, as an additive to gasoline. Additionally, the ethanol can be burned in combustion generators to produce electricity.

In a specific embodiment, the method comprises loading an ethanologenic microorganism that has been immobilized, into a column of the subject system; mixing water and a carbohydrate in a feed tank that is attached to the column; and using a pumping apparatus, or gravity, to continuously transfer the water and carbohydrate mixture from the feed tank and over the immobilized microorganism at a consistent dilution rate, wherein the system is operated for an appropriate amount of time to produce an end product comprising 6-15 g/L of ethanol.

The method can further comprise distilling the end product to a desired purity and/or alcohol content.

In one embodiment, the microorganism is immobilized in and/or on alginate beads. In one embodiment, the microorganism is immobilized in the pores and on the surfaces of macroporous, sterile beads made of glass or plastic. In one embodiment, the microorganisms are immobilized onto a line or fiber suspended from the top of the column to the bottom of the column. In certain embodiments, the substrate to which the yeast is immobilized has been functionalized with an antibody, or other linker, to help facilitate immobilization, yet maintained biological activity, of the yeast.

Preferably, the continuous circulation of liquid into and out of the column allows for the continual removal of the ethanol-containing end product so as not to exceed a concentration of 6-15 g/L. Advantageously, this reduces growth inhibition of the microorganisms by the ethanol and facilitates continuous operation of the system.

Advantageously, the subject invention provides for conditions in which a high conversion rate can be achieved. That is, the subject methods and systems allow for the conversion of carbohydrate sources into ethanol in large quantities quickly and efficiently, for example, in 36 hours.

Furthermore, operation of the subject invention produces little to no residual waste. In certain embodiments, any yeast solids (e.g., yeasts and alginate) that are left over from the conversion process can be used as, for example, livestock feed, compost material, or as an agricultural soil amendment.

Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state or inactive or immobilized. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells may be absent, or present at, for example, a concentration of 1×10⁴, 1×10⁵, 1×10⁶, 1×10⁷, 1×10⁸, 1×10⁹, 1×10, or 1×10¹¹ or more cells per milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, such as plant hormones, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, for example, filtering, centrifugation, lysing, drying, purification and the like.

As used herein, “harvested” refers to removing some or all of the microbe-based composition from a growth vessel.

As used herein, an “isolated” or “purified” nucleic acid molecule, polynucleotide, polypeptide, protein or organic compound, such as a small molecule, is substantially free of other compounds, such as cellular material, with which it is associated in nature. As used herein, reference to an “isolated” strain means a microbial strain that is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “metabolite” refers to any substance produced by metabolism or a substance necessary for taking part in a particular metabolic process. A metabolite can be an organic compound that is a starting material (e.g., glucose), an intermediate (e.g., acetyl-CoA) in, or an end product (e.g., n-butanol) of metabolism. Examples of metabolites can include, but are not limited to, enzymes, toxins, acids, solvents, alcohols, proteins, carbohydrates, vitamins, minerals, microelements, amino acids, polymers, and surfactants.

As used herein, a “plurality” or a “series” refers to any whole number greater than one.

As used herein, “alginate” means any of the conventional salts of algin, a polysaccharide of marine algae which may be polymerized to form a matrix for use within the growth chamber of the bioreactor. The salts of algin can include, but are not limited to, any metal salt such as sodium, magnesium, calcium, etc. The alginate can further include a composition of gluronic and mannuronic acids, and preferably has a low viscosity.

As used herein, the term “carbohydrate” refers to any carbohydrate that a microorganism can utilize, metabolize, convert and/or ferment. Carbohydrates can include monosaccharides, disaccharides, oligosaccharides, and other sugars such as glucose, xylose, galactose, arabinose, mannose, sucrose, fructose, and/or maltose. In preferred embodiments, the carbohydrates used according to the present invention are locally derived, or locally sourced, meaning they are obtained from sources that are within 10, 15, 20, 25, 30, 35, 40, 45 or 50 miles from the site of production of and/or use of the products of their conversion. For example, the carbohydrates can be derived (e.g., using hydrolysis) from locally sourced wort, sugarcane, molasses, sugar beets, fruit juice, sugar syrup, hops, barley, wheat, rye, rice, fruits (e.g., grapes, berries, apples), agave, starch syrup, grains, potatoes, other food crops, or can be any other hydrolysate from plant material capable of conversion by ethanologenic microorganisms. In one embodiment, if, for example, the locally sourced carbohydrate is in a form that cannot be converted into a desired by-product by a microorganism, the carbohydrate source can be pre-processed, for example, using enzymes to produce sugar hydrolysates.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ethanol Production System Design and Operation

In preferred embodiments, the subject invention provides a portable system for the production of bioethanol. In particular, the system comprises one or more biological reactors for conversion of carbohydrates into an end product, wherein the end product comprises an alcohol, e.g., ethanol. The system further comprises a distilling apparatus for distilling, or purifying, the ethanol to a distilled alcohol product having a desired ethanol concentration and/or purity. The distilled alcohol product produced according to the subject invention can be used in a variety of application, including, for example, as an additive for gasoline or for generating electricity.

The entire system can be housed and transported in a protective container measuring, for example, 10 square feet to 50 square feet, more preferably 20 square feet to 40 square feet. The protective container can comprise handles and optionally, wheels, for moving and maneuvering the system.

In certain embodiments, the container can be fashioned to withstand harsh environmental conditions such as those in remote geographic locations or on military bases. For example, in certain embodiments, the container can be made of material that is fire proof, explosion proof, ice proof, wind proof, or water proof. In one embodiment, the container is an explosion proof enclosure, made of fiberglass, acrylic, plastic, steel, aluminum, and/or alloys or combinations thereof.

The system can be configured on, for example, a trailer or a truck bed. The system can also be designed to be portable by means such as a pickup truck, a flatbed trailer, a semi-trailer, a Humvee, an SUV, a forklift, or even a train, tank, cargo plane, helicopter or boat. As a result, the systems can be operated at or near a site where fuel alcohol is needed for fuel or electricity production. For example, the systems can he operated within 1, 5, 10, 15, 20, 25, 30, 35, 40, 45 or 50 miles from the site where ethanol is needed.

The system can comprise a platform and/or a frame for supporting the various components (including, e.g., the container, column, pump, feed tank, distiller, etc.). In one embodiment, the various components of the system can be secured in place onto the platform. The platform can have wheels with optional breaking mechanisms attached thereto, and handles for moving and maneuvering the system. The platform can comprise a hook or hook or tongue coupler/trailer coupler, or other mechanism for attaching the platform to, for example, a tow hitch. Thus, the system can be configured to be pulled or towed via automobile. Furthermore, the protective container can comprise a locking mechanism for securing the container onto the platform so that it will not move or fall off of the platform during transport of the system.

Even further, the systems can be useful in rural or secluded areas, where transmission of power is difficult due to the remoteness of the area or the distance from the nearest power plant or electricity distribution system. For example, the systems can be used to produce ethanol for use in local and/or household generation of electricity in cities and towns located in remote, rural and/or secluded areas or communities.

In one embodiment, the system can be operated in an area or community that is at least 10 miles from an electricity distribution system. In one embodiment, the system can be operated on a military base, or in a military conflict zone. In one embodiment, the system can be operated on an island, or in the mountains. The system can also be operated in farming communities, for example, for households and for powering gasoline-powered agricultural equipment.

Additionally, the portable systems can be operated at or near a site where ethanol is needed, for example, not more than 50 miles away from a site where electricity or fuel is needed. The systems can be useful in rural or secluded areas, where transmission of power is difficult due to the remoteness of the area or the distance from the nearest power plant. For example, the systems can be used to produce ethanol for use in local or household generation of electricity in rural cities and towns.

In one embodiment, the portable systems of the present invention provide biological reactors for continuous conversion of carbohydrates into ethanol. Advantageously, the systems of the present invention can be scaled depending on the intended use. For example, in some embodiments, the system comprises a plurality of biological reactors working simultaneously to produce ethanol in mass quantities.

In certain embodiments, the systems utilizes ethanologenic organisms to convert carbohydrates into ethanol. In preferred embodiments, the systems utilize Wickerhamomyces anomalus or Saccharomyces cerevisiae yeasts, immobilized in and/or on a bead or some other medium for immobilizing yeasts in a resting state.

In certain embodiments, the biological reactors of the subject systems can comprise a column, wherein the column is attached to a feed tank containing a mixture of a carbohydrate and water. The column can be any known column having a high vertical to horizontal ratio, for example, a tube of a Winogradsky column.

The column can be made of glass, polymers, metals, metal alloys, plastic, or combinations thereof. The column can be transparent or opaque. Prior to conversion, the column may be disinfected or sterilized. The size of the column can be adjusted, depending on the amount of end product desired. For example, the column can range from 5 liters to 2,000 liters or more, more typically from 10 liters to 1,000 liters. In a specific embodiment, the column is 10 liters.

Preferably, the dimensions of the column comprise a high vertical to horizontal ratio, for example, the ratio of height to diameter is at least 5:3, more preferably 10:1, and even more preferably 20:1.

The column can further comprise a vent or an off gas orifice for releasing carbon dioxide and other gases produced during conversion.

In preferred embodiments, the column is loaded with immobilized yeast cells. In one embodiment, the microorganism is immobilized in and/or on alginate beads. In one embodiment, the microorganism is immobilized in the pores and on the surfaces of microporous, sterile beads made of glass or plastic. In one embodiment, the microorganisms are immobilized onto a line or fiber suspended from the top of the column to the bottom of the column. In certain embodiments, the substrate to which the yeast is immobilized has been functionalized with an antibody, or other linker, to help facilitate immobilization, yet maintained biological activity, of the yeast.

Preferably, the yeast cells are immobilized in and/or on alginate beads. The beads can either be prepared ahead of time or prepared on site. Preferably, preparation of the alginate beads comprises cultivating and concentrating the desired yeast cell line according to known methods. In one embodiment, the concentrated yeast cell suspension is obtained through cultivation processes ranging from small (e.g., lab setting) to large (e.g., industrial setting) scales. These cultivation processes include, but are not limited to, submerged cultivation/fermentation, solid state fermentation (SSF), and combinations thereof.

Sodium alginate (3% w/v) sterile solution is then mixed with the cell suspension in a ratio of, for example, 5:1, to produce an alginate-yeast mixture. The skilled artisan would understand that this ratio can be optimized depending on any number of factors, such as number of yeast cells or size of the column.

The alginate-yeast mixture is then showered into a 2-3% sterile calcium chloride solution using, for example, a dropping device or a pipette. On contact with the calcium solution, the alginate polymerizes to form beads, immobilizing the yeasts within and on the outer surface of the beads. The beads can then be strained from the solution, washed, and then packed into a column reactor of the subject system.

In one embodiment, the column is connected to a feed tank via tubing or piping. The feed tank can contain a solution comprising a carbohydrate and water. In certain embodiments, the carbohydrate is a sugar. In specific embodiments, the carbohydrate is glucose derived from a local source. Preferably, the concentration of carbohydrate in the carbohydrate-water solution is maintained at from 200 to 300 g/L, more preferably from 200 to 250 g/L.

Pressure is then used to continuously transfer the carbohydrate-water mixture from the feed tank, through a tube or pipe, into the column, and over the immobilized yeast, so as to, preferably, achieve a consistent dilution rate throughout the conversion process. In one embodiment, the carbohydrate-water mixture flows into the column through a tube or pipe at the bottom of the column and flows out through a tube or pipe at the top. In one embodiment, the carbohydrate-water mixture flows into the column through a tube or pipe at the top of the column and flows out through a tube or pipe at the bottom.

In one embodiment, the system utilizes gravity to transfer the carbohydrate-water mixture from the feed tank and through the column, wherein the feed tank can be in an elevated position relative to the column.

In another embodiment, the system utilizes a pump to transfer the carbohydrate-water mixture. The pump can be, for example, a dosing pump, a peristaltic pump, or a centrifugal pump. The pump can be scaled depending on the size of the reactor column and the amount of liquid therein. Preferably, the pump is scaled to be suitable for establishing a consistent dilution rate of about 0.2 to 0.3 total volume per hour. In preferred embodiments, the pump operates continuously throughout the process of conversion. The pump can be controlled using, for example, a variable frequency motor so that flow rates can be properly adjusted with any change in electrical current.

Advantageously, the use of immobilized yeasts according to the present system allows for stable, long running operation of biological reactors, as they allow for maintained cell viability over extended periods of time. Additionally, a higher yeast cell concentration can be achieved. As the liquid carbohydrate solution is passed through the column packed with immobilized yeast, the number of yeast cells that come into contact with the carbohydrate in the reactor is greatly increased. This greater contact results in an increased speed of conversion.

In certain embodiments, the systems further comprise a distilling apparatus for distilling and collecting ethanol from the products of conversion. The distilling apparatus can be a distiller, a still, a beer column, or any other system or apparatus known in the art of alcohol production. The conversion products can be transferred directly from the column to the distiller, for example, by piping or tubing, or can be collected in an interim vessel or container and then manually added to the distilling apparatus. Preferably, the conversion products are continually removed from the column so as not to inhibit microbial survival (e.g., by keeping ethanol concentration at or below 6-12 g/L or 6-15 g/L).

In one embodiment, the column reactors have functional controls/sensors or may be connected to functional controls/sensors to measure important factors in the conversion process, such as pH, oxygen, pressure, temperature, humidity, viscosity and/or microbial density and/or metabolite concentration.

In one embodiment, each column reactor has its own controls and measuring systems for at least temperature and pH. In addition to monitoring and controlling temperature and pH, each reactor may also have the capability for monitoring and controlling, for example, dissolved oxygen, agitation, foaming, purity of microbial cultures, production of desired metabolites and the like. Monitoring of these parameters can occur remotely, for example using a tablet, smart phone, or other mobile computing device capable of sending and receiving data wirelessly.

In one embodiment, equipment used in the method and conversion process are sterilized. The equipment such as the column reactor may be separated from, but connected to, a sterilizing unit, e.g., an autoclave. The cultivation equipment may also have a sterilizing unit that sterilizes in situ before starting the conversion process, e.g., by using steam.

In one embodiment, before conversion, the column reactor can be washed with a hydrogen peroxide solution (e.g., from 2.0% to 4.0% hydrogen peroxide; this can be done before or after a hot water rinse at, e.g., 80-90 degrees Celsius) to prevent contamination. In addition, or in the alternative, the column can be washed with a bleach solution and a hot water rinse. The culture medium components can also be temperature decontaminated and/or hydrogen peroxide decontaminated (potentially followed by neutralizing the hydrogen peroxide using an acid such as HCl, H₂SO₄, etc.).

In one embodiment, minimal sterilization, for example, simply soap and water, is needed to prevent contamination of the subject system. This is due to the antimicrobial properties of many killer yeast strains, which helps prevent growth of undesirable microorganisms. For example, in one embodiment wherein Wickerhamomyces anomalus is the chosen ethanoligenic microorganism, the system can be self-sterilizing.

The system can be used as a batch reactor (as opposed to a continuous reactor). Advantageously, the system can be scaled depending on its intended use.

The system can also be adapted to ensure maintaining an appropriate conversion temperature. For example, the system can be insulated so the conversion process can remain at appropriate temperatures in low temperature environments. Any of the insulating materials known in the art can be applied including fiberglass, silica aerogel, ceramic fiber insulation, etc.

Additionally, the outside of the system can be reflective to avoid raising the system temperature during the day. Furthermore, a cooling system can be added to the reactor that includes, for example, one or more of a cooling jacket and a cooling heat exchanger. Cooling water can be passed through the jacket, exchanging heat with ambient air and then recirculating through the cooling system. For extreme environments, the system can include refrigeration and cooling coils within the reactor, a jacket surrounding the reactor, or heat exchangers connected to the pump.

The system can utilize an electric heater. However, for larger applications where heat is required, steam or hydrocarbon fuel can be utilized. A steam input and/or a steam source can be connected to one or more of a steam injector, a steam jacket, and a steam heat exchanger. In addition, steam can be directly injected into the pumping mechanism. A steam heat exchanger can be placed inside the reactor, steam can condense within the tubes of the heat exchanger, and then be expelled. The steam heat exchanger can be a closed system that does not mix water or steam into the reactor.

In some embodiments, the system can operate continuously, for several hours, days, or months. In one embodiment, the system operates for over one month. In another embodiment, the system operates for 1 hour to 120 hours, or about 12 to about 96 hours. In specific embodiments, the subject system can produce high concentrations of ethanol, e.g., 50 to 70%, or 60 to 80%, or 70 to 90% alcohol by volume, in as little as 36 hours of continuous operation.

In some embodiments, the system of the subject invention can be powered primarily by generators located, for example, in trucks used to carry the contained system from point to point. In one embodiment, the generators can be powered by, for example, diesel fuel. In one embodiment, bioethanol can supplement or replace the fuel used to power the generators. In certain embodiments, the bioethanol used to supplement or replace the fuel is a portion of the product of conversion according to operation of the subject systems. Thus, the system can be partially or entirely self-sustaining.

In some embodiments, solar panels can be installed on top of, or otherwise near, the housing container to provide supplemental energy. In yet another embodiment, supplemental energy can be provided by wind turbines, which can also be portable.

Advantageously, operation of the subject invention produces little to no residual waste. In certain embodiments, any solids (e.g., biomass and alginate) that are left over from the conversion process can be used as, for example, livestock feed, compost material, or as an agricultural soil amendment.

In one embodiment, the subject system can be part of a stand-alone power system (SAPS) or remote area power supply (RAPS). SAPSs and RAPSs are electricity generating systems that are operated off the electrical grid for locations that are not fitted with an electricity distribution system. SAPSs and RAPSs can include multiple components, e.g., for generating electricity, storing energy, and regulating energy.

Methods of Bioethanol Production

In one embodiment, methods are provided for producing bioethanol using the systems according to the subject invention. Ethanol produced according to the subject methods can be used to supplement existing fuel sources, for example, as an additive to gasoline. Additionally, the ethanol can be burned in combustion generators to produce electricity.

In a specific embodiment, a method is provided for converting a carbohydrate into ethanol, the method comprising loading an immobilized ethanologenic microorganism into a column of the subject system; mixing water and a carbohydrate in a feed tank that is attached to the column; and using a pumping apparatus, or gravity, to continuously transfer the water and carbohydrate mixture from the feed tank, over the immobilized microorganism, and through the column at a consistent dilution rate, wherein the system is operated for an appropriate amount of time to produce an end product comprising 6-12 g/L or 6-15 g/L of ethanol.

In one embodiment, the microorganism is immobilized in and/or on alginate beads. In one embodiment, the microorganism is immobilized in the pores and on the surfaces of macroporous, sterile beads made of glass or plastic. In one embodiment, the microorganisms are immobilized onto a line or fiber suspended from the top of the column to the bottom of the column.

The method can comprise immobilizing the microorganism prior to loading into the column. For example, the microorganism can be immobilized at the site where the system is being operated, or it can occur elsewhere, in which case, the immobilized microbes are shipped to the site where the system is being operated.

Preferably, the continuous circulation of liquid into and out of the column allows for the continual removal of the ethanol-containing end product so as not to exceed a concentration of 6-15 g/L. Advantageously, this reduces growth inhibition of the microorganisms by the ethanol and facilitates continuous operation of the system.

The microorganisms utilized according to the subject methods can be, for example, bacteria, yeast, fungi or multicellular organisms. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In preferred embodiments, the microbes are ethanologenic yeasts. Yeasts suitable according to the present invention include, but are not limited to Wickerhamomyces (e.g., W. anomalus), Saccharomyces (e.g., S. cerevisiae and S. uvarum), Pichia (P. anomala, P. guilliermondii, P. occidentalis, P. kudriavzevii), Candida (e.g., C. utilis, C. arabinofermentans, C. diddensii, C. sonorensis, C. shehatae, C. tropicalis, and C. boidinii).

Other suitable organisms include, but are not limited to strains of Zymomonas, Hansenula (e.g., H. polymorpha and H. anomala), Kluyveromyces (e.g., K. fragilis and K. marxianus), Schizosaccharomyces (e.g., S. pombe), Clavispora (e.g., C. lusitaniae and C. opuntiae), Pachysolen (e.g., P. tannophilus), Bretannomyces (e.g., B. clausenii), Saccharophagus (e.g., S. degradans), as well as any strain that falls within the category of “killer yeast.”

As used herein, “killer yeast” means a strain of yeast characterized by its secretion of toxic proteins or glycoproteins, to which the strain itself is immune. The exotoxins secreted by killer yeasts are capable of killing other strains of yeast, fungi, and bacteria. Such yeasts can include, but are not limited to, strains of the genera Wickerhamomyces (e.g., W. anomalus), Pichia (e.g., P. anomala, P. guielliermondii, P. occidentalis, P. kudriavzevii), Hansenula, Saccharomyces, Hanseniaspora, (e.g., H. uvarum), Ustilago maydis, Debaryomyces hansenii, Candida, Cryptococcus, Kluyveromyces, Torulopsis, Ustilago, Williopsis, Zygosaccharomyces (e.g., Z. bailii), and others.

In one embodiment, the microorganism is a strain of Wickerhamomyces anomalus or a mutant thereof. Wickerhamomyces anomalus, also known as Pichia anomala and Hansenula anomala, is capable of growing on a wide range of carbon sources at low pH, under high osmotic pressure and in aerobic or microaerophilic conditions, allowing for its survival in a wide range of environments.

In specific embodiments, the subject invention provides the use of Saccharomyces cerevisiae yeast strains and mutants thereof.

In one embodiment, a single type of microbe is utilized in the subject system. In alternative embodiments, multiple microbes, which can be grown together without deleterious effects on each other or the resulting product, can be utilized in a single vessel. There may be, for example, 2 to 3 or more different microbes in a single vessel at the same time.

In some embodiments, the carbohydrate utilized in the subject invention is a sugar. In specific embodiments, the carbohydrate is locally derived, or locally sourced, glucose or sucrose. The concentration of glucose or sucrose in the carbohydrate-water solution is preferably from 200 to 300 g/L, even more preferably, 200 to 250 g/L.

Advantageously, the subject invention provides for conditions in which a high conversion rate can be achieved. That is, the subject methods and systems allow for the conversion of carbohydrate sources into ethanolic byproducts in large quantities quickly and efficiently, for example, in as little as 36 hours.

Additionally, the subject invention allows for continuous, uninterrupted production of yeast products over extended periods of time. For example, the biological reactors can be operated continuously, 24 hours a day, for several days or even months at a time. This is, in part, due to high yeast survival rates. For example, a yeast survival rate of 95% over the course of one month can be achieved using the subject systems, thus reducing the number of times the system must be re-inoculated with yeast cells.

In preferred embodiments, conversion is allowed to proceed for as long as desired, as long as concentration of ethanol in the reactor does not exceed 12-15 g/L. Above this concentration, conversion will decline and eventually halt due to yeast inhibition. Thus, the method can comprise periodically measuring the ethanol concentration within the reactor and continually or as needed, removing the ethanol-containing end product so as not to exceed an ethanol concentration of 12-15 g/L.

Advantageously, this reduces growth inhibition of the microorganisms by the ethanol and facilitates continuous operation of the system.

In one embodiment, the method further comprises the step of distilling, or purifying, the ethanol and other alcohols from the resulting end product. Distillation is the process by which alcohol is purified or removed from other diluting components, thereby increasing the percentage of alcohol in the final solution. Typically, distillation involves vaporizing a liquid containing alcohol, then cooling the vapor and collecting the resulting condensate liquid, i.e., the distilled alcohol.

In certain embodiments, distillation is achieved using a distilling apparatus. The apparatus can be a distiller, a still, a beer column, or any other system or apparatus known for use in alcohol production.

In specific embodiments of the subject method, distilling the ethanol from the end product comprises adding the end product to a distilling apparatus, distilling the end product to produce a distilled alcohol product, and collecting the distilled alcohol product. In one embodiment, the end product can be transferred directly from the column to the distilling apparatus, for example, using piping. In another embodiment, the end product can be collected in an interim vessel or container and manually transferred to the distilling apparatus. In one embodiment, the end product can be a distilled alcohol product that is 50-70%, 60-80%, or 70-90% alcohol by volume.

Following distillation, the distilled alcohol product can be collected in a tank for processing according to desired uses. In certain embodiments, the distilled alcohol product is ethanol. In one embodiment, the ethanol is mixed with gasoline as a supplemental fuel source. In another embodiment, the ethanol is burned using a combustion generator and used for producing electrical energy. In a further embodiment, the distilled alcohol product can be filtered and pasteurized for use in a consumable alcoholic beverage.

It is not intended that the methods provided herein be necessarily limited to the production of any specific end product. In some embodiments, end products include fuel alcohols or precursor industrial chemicals. For example, in some embodiments, conversion products include precursor industrial chemicals such as alcohols (e.g., ethanol, propanol, methanol and/or butanol); organic acids (e.g., butyric acid, citric acid, acetic acid, itaconic acid, lactic acid, and/or gluconic acid); ketones (e.g., acetone); amino acids (e.g., glutamic acid); gases (e.g., H₂ and/or CO₂); antimicrobials (e.g., penicillin, sophorolipids and/or tetracycline); enzymes; vitamins (e.g., riboflavin, B₁₂, and/or beta-carotene); and/or hormones.

In some embodiments, the end product comprises a fuel alcohol. Suitable fuel alcohols are known in the art and include, but are not limited to lower alcohols such as methanol, ethanol, butanol and propyl alcohols. In preferred embodiments, the end product comprises ethanol.

As described above, in some embodiments, the methods described herein can be used for converting biomass to an energy product (e.g., an alcohol such as ethanol) and/or other products that result from the conversion process. In such cases, the biomass will be exposed to conditions suitable for such a conversion. Exemplary conditions can include, e.g., at least biomass and one or more microorganisms capable of converting the biomass to energy (e.g., an alcohol) in an environment suitable for those organisms to function. This conversion process can be allowed to proceed to a point where at least a portion of the biomass is converted to energy (e.g., ethanol) and/or other products that result from the process and/or to a point where all (e.g., essentially all) of the materials are converted to energy (e.g., ethanol) and/or other products that result from the conversion process. For example, at least about 10, 20, 30, 40, 50, 60, 70, 80, 90, 95, 98, 99, 99.5, or 100% of the materials exposed to suitable conditions is converted to energy (e.g., ethanol) and/or other products that result from the conversion process.

Advantageously, the method does not require complicated equipment or high energy consumption. The microorganisms of interest can be cultivated at small or large scale on site and used directly for their intended purpose. Similarly, the microbial metabolites, for example, ethanol, can also be produced at large quantities at the site of need.

In certain embodiments, the methods of the subject invention can be carried out in remote, rural and/or secluded areas or communities, e.g., in areas or communities that are at least 10 miles from an electricity distribution system. In one embodiment, the methods can be carried out on a military base, or in a military conflict zone. In one embodiment, the methods can be carried out on an island, or in a community in the mountains, or in a farming community.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1

### Immobilization of Yeast Cells in Alginate Beads

Yeast cells can be immobilized in and/or on alginate beads. Preparation of the alginate beads can comprise cultivating and concentrating the desired yeast cell line according to known methods. Once the yeast reaches the stationary phase, it is collected and subjected to purification, e.g., by microfiltration and/or centrifugation.

The cell suspension is then mixed with 2-5% sodium alginate (preferably 3% w/v) sterile solution in a ratio of, for example, 5:1, to produce an alginate-yeast mixture. The skilled artisan would understand that this ratio can be optimized depending on any number of factors, such as number of yeast cells or size of the column.

The alginate-yeast mixture is then dropped using a standard pipette or titration apparatus into a 2-3% CaCl₂ solution. Beads then form, which can be removed, washed, and placed into a reactor column of the subject invention.

It should be understood that the examples and embodiments described herein are for illustrative purposes only and that various modifications or changes in light thereof will be suggested to persons skilled in the art and are to be included within the spirit and purview of this application and the scope of the appended claims.

1. A system for producing bioethanol, wherein the system comprises:
a column loaded with an immobilized ethanologenic microorganism;
a feed tank having water and a carbohydrate therein, wherein the feed tank is attached to the column via tubing or piping; and
a distilling apparatus;
wherein the column, feed tank, and distilling apparatus are enclosed within a protective container with abase 10 square feet to 50 square feet, and
wherein the ethanologenic microorganism is Wickerhamomyces anomalus.

2. The system of claim 1, wherein the distilling apparatus is a distiller, a still, or a beer column.

3. The system of claim 1, wherein the protective container is made of materials that are fire proof, explosion proof and/or waterproof.

4. The system of claim 3, wherein the protective container is made of stainless steel, aluminum, alloys thereof, or combinations thereof.

5. The system of claim 1, further comprising a platform, wherein the column, feed tank, distilling apparatus and pump apparatus are secured onto the platform, and wherein the protective container comprises a locking mechanism for securing the container onto the platform.

6. The system of claim 5, wherein the platform comprises wheels and handles for moving and maneuvering the system.

7. The system of claim 5, wherein the platform comprises a hook or tongue coupler for pulling or towing the system.

8. (canceled)

9. The system of claim 1, wherein the mixture of carbohydrate and water in the feed tank has a concentration of carbohydrate at 200-300 g/L.

10. The system of claim 8, wherein the carbohydrate is glucose, sucrose, wort, sugarcane, molasses, sugar beets, fruit juice, sugar syrup, starch syrup, grains, fruit or hydrolysates thereof sourced from a location not more than 50 miles away.

11. The system of claim 1, wherein the system utilizes gravity to transfer the carbohydrate-water mixture from the feed tank and through the column, wherein the feed tank is positioned in an elevated position relative to the column.

12. The system of claim 1, further comprising a pumping apparatus, wherein the pumping apparatus is a dosing pump, a peristaltic pump, or a centrifugal pump.

13. The system of claim 1, wherein the pumping apparatus is used to run the mixture of carbohydrate and water from the feeding tank through the column and over the immobilized yeasts such that a consistent dilution rate is achieved throughout operation of the system.

14. The system of claim 1, wherein the column is used for conversion of the carbohydrate to produce an end product comprising ethanol, and wherein the distilling apparatus is used to distill the ethanol to a distilled alcohol product having a desired ethanol concentration and/or purity.

15. The system of claim 1, wherein the microorganism is immobilized in and/or on alginate beads.

16. The system of claim 1, wherein the system is operated not more than 50 miles from where ethanol is needed.

17. The system of claim 1, wherein the system is operated on a military base or at a military station.

18. The system of claim 1, wherein the system is operated in an area or community that is at least 10 miles from an electricity distribution system.

19. The system of claim 1, powered by a diesel engine, a solar panel and/or wind turbine.

20. The system of claim 1, wherein the system is part of a stand-alone power system (SAPS) or remote area power supply (RAPS).

21. A method for producing bioethanol, wherein the method comprises:
loading an immobilized ethanologenic microorganism into a column of a system of claim 1;
mixing water and a carbohydrate in a feed tank that is attached to the column via tubing or piping, wherein the concentration of the carbohydrate is 200 to 250 g/L; and
using a pumping apparatus, or gravity, to continuously transfer the water and carbohydrate mixture from the feed tank, through the column, and over the immobilized microorganism at a consistent dilution rate,
wherein the system is operated for an appropriate amount of time to produce an end product comprising 6-15 g/L of ethanol, and
wherein the ethanologenic microorganism is Wickerhamomyces anomalus.

22. The method of claim 21, wherein the carbohydrate is sourced from locally derived wort, sugarcane, molasses, sugar beets, fruit juice, sugar syrup, starch syrup, grains, fruits or hydrolysates thereof.

23. The method of 22, wherein the carbohydrate is a source of glucose.

24. The method of claim 22, wherein the carbohydrate is a source of sucrose.

25-26 (canceled)

27. The method of claim 21, further comprising the step of distilling the ethanol from the end product to produce a distilled alcohol product.

28. The method of claim 27, wherein the distilled alcohol product is ethanol at a concentration of 70 to 90% alcohol by volume.

29. The method of claim 27, further comprising processing the distilled alcohol product for use as a fuel additive or for powering an electrical combustion generator.

30. The method of claim 21, carried out within 50 miles of where ethanol is needed.

31. The method of claim 21, carried out in an area or community that is remote, rural or secluded, wherein the area or community is at least 10 miles from an electricity distribution system.

32. The method of claim 31, carried out on a military base or at a military station.

33. The method of claim 31, wherein the area or community is an island, mountain community, or farming community.

34. The method of claim 21, wherein the microorganism is immobilized in and/or on alginate beads.
